SEQUENCE LISTING (6 sequences)

SEQ ID NO: 1 - DNA, 32 bp, Artificial Sequence
  Smooth muscle alpha actin forward PCR primer with restriction site
acaacaatcgataacagctggtcatggctgta

SEQ ID NO: 2 - DNA, 32 bp, Artificial Sequence
  smooth muscle alpha actin promoter PCR primer
tgttgtaccggtgcatgaacccagccaaatcc

SEQ ID NO: 3 - DNA, 32 bp, Artificial Sequence
  forward PCR primer for amplification of myosin heavy chain promoter
acaacaatcgatagcctgtgcggagcagctca

SEQ ID NO: 4 - DNA, 32 bp, Artificial Sequence
  reverse primer for PCR amplificaiton of myosin heavy chain promoter
acaacagctagcgatggagagctcggatctga

SEQ ID NO: 5 - DNA, 29 bp, Artificial Sequence
  rat SMaA promoter for cloning rat SMaA in fron of promoterless EGFP
cctctagaccacggtccttaagcatgata

SEQ ID NO: 6 - DNA, 35 bp, Artificial Sequence
  SMaA reverse PCR primer for cloning SMaA promoter in front of promoterless EGFP
aactcgagccttacttgtacagctcgtccatgccg

(19) United States
(12) Patent Application Publication
(10) Pub. No.: US 2010/0273231 A1
(43) Pub. Date: Oct. 28, 2010

(54) Multipotent mesenchymal stem cells from human hair follicles

(76) Inventors: Stelios T. Andreadis, East Amherst, NY (US); Jinyu Liu, Amherst, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/590,427

(22) Filed: Nov. 6, 2009

Related U.S. Application Data
(63) Continuation-in-part of application No. 11/523,474, filed on Sep. 19, 2006, now abandoned.
(60) Provisional application No. 60/718,813, filed on Sep. 20, 2005.
(60) Provisional application No. 61/111,978, filed on Nov. 6, 2008.

(51) Int. Cl. C12N 11/02; C12N 5/077

(52) U.S. Cl. 435/177; 435/366

(57) ABSTRACT

The present invention is directed to an enriched preparation of multipotent mesenchymal cells obtained from human hair follicle stem cells, preferably adult human hair follicle cells. These cells can be differentiated into adipogenic, chondrogenic and osteogenic cells as well as smooth muscle cells. The present invention is also directed to a method of producing a tissue-engineered vascular vessel containing the preparation of isolated smooth muscle cells or progenitors thereof. The resulting tissue-engineered vascular vessel and a method of producing a tissue-engineered vascular vessel for a particular patient are also disclosed.

This application claims priority to U.S. Provisional Patent Application No. 61/111,978, filed on Nov. 6, 2008, and is a continuation-in-part of U.S. Non-provisional patent application Ser. No. 11/523,474, filed on Sep. 19, 2006, which in turn claims priority to U.S. Provisional Patent Application No. 60/718,813, filed Sep. 20, 2005, the disclosures of all of which are hereby incorporated by reference in their entirety.

### FIELD OF THE INVENTION

The present invention relates generally to multipotent stem cells, and particularly to a preparation of stem cells isolated from human hair follicles. The human hair follicle stem cells are capable of differentiating toward a mesenchymal path as well as a myogenic path.

### BACKGROUND OF THE INVENTION

Stem cells have tremendous potential as an autologous, non-immunogenic cell source for tissue regeneration. Specifically, adult stem cells provide a promising alternative to embryonic stem cells and can be isolated from the same patient, which avoids immune rejection and long-term immunosuppression. For example, bone marrow-derived stem cells have high proliferation potential, can home into sites of vascular injury where they differentiate into vascular cells, and adult stem cells are not compounded by the ethical considerations of embryonic stem cells and they are readily available for research. However, the process of obtaining bone marrow from individuals involves tedious and uncomfortable invasive procedures.

An alternative source of stem cells is the hair follicle. It is one of the few structures in the body with the ability to undergo cycles of degeneration and regeneration throughout life and provides a source which is easily accessible. Isolation of stem cells from hair follicle involves obtaining hair follicles and culturing certain portions of the hair follicle.

While stem cells can be coaxed into differentiation toward various phenotypes depending upon the source of the cells, the ability to differentiate stem cells into smooth muscle cells is of particular interest for the construction of tissue engineered blood vessels. However, given the generally accepted difficulties in culturing adult cells as well as the limited replicative capacity of adult cells in culture, harvesting of hair follicle stem cells from adults that could then be differentiated into functional smooth muscle cells has not been heretofore reported.

Accordingly, there continues to be a need for easily accessible, autologous source of progenitor vascular cells for development of cell therapies for cardiovascular disease.

### SUMMARY OF THE INVENTION

One aspect of the present invention is directed to a method of isolating a multipotent population of cells from hair follicle cells. In one embodiment, the hair follicle cells are human hair follicle cells. In another embodiment, the hair follicle cells are isolated from an adult individual. In yet another embodiment, the hair follicle cells are isolated from an adult human.

Another aspect of the invention is directed to a method for preparing enriched preparations of mesenchymal stem cells from hair follicle cells. The mesenchymal cells can be differentiated into chondrocytes, adipocytes, osteoblasts, smooth muscle cells, endothelial like cells, cardiomyocytes and myocytes. The method comprises exposing the enriched populations of mesenchymal hair follicle stem cells to incubating media suitable for coaxing differentiation of cells toward the desired lineage.

Another aspect of the invention provides a composition enriched for mesenchymal stem cells which can be differentiated into adipogenic, chondrogenic, osteogenic and functional contractile cells.

Another aspect of the invention is directed to isolating smooth muscle cells or progenitors thereof from a mixed population of hair follicle stem cells or from an enriched preparation of mesenchymal cells. This method involves incubating the mixed population of cells or the enriched mesenchymal cells with the appropriate medium to stimulate differentiation into smooth muscle cells. If desired, selection of the differentiated cells can be carried out by using an enhancer/promoter which functions in the smooth muscle cells or progenitors thereof. A nucleic acid molecule encoding a marker protein under control of the enhancer/promoter is introduced into the cells. The smooth muscle cells or progenitors thereof are allowed to express the marker protein. The smooth muscle cells or progenitors thereof are separated from the mixed population of cells based on expression of the marker protein.

Another aspect of the present invention is directed to a preparation comprising isolated smooth muscle cells or progenitors thereof prepared from a mixed population of hair follicle stem cells or from enriched mesenchymal cells, where the smooth muscle cells or progenitors thereof constitute at least 90, 91, 92, 93, 94, 95, 96, 97, 98, 99 or 100% of said preparation.

A further aspect of the present invention is directed to a method of producing a tissue-engineered vascular vessel. This method involves providing a vessel-forming matrix or scaffold, and isolated smooth muscle cells or progenitors thereof. The vessel-forming scaffold mixture is molded into a tubular shape. The tubular shaped scaffold is incubated in a medium suitable for growth of the cells under conditions effective to produce a tissue-engineered vascular vessel. In one embodiment, the scaffold comprises a fibrin mixture containing fibrinogen and thrombin. In another embodiment, the scaffold comprises subintestinal submucosa (SIS). In one embodiment the smooth muscle cells and/or the scaffold material is/are autologous to the patient

Yet another aspect of the present invention is directed to a tissue-engineered vascular vessel containing a scaffold and the preparation of isolated smooth muscle cells or progenitors thereof.

### BRIEF DESCRIPTION OF THE DRAWINGS

FIG. 1A-B: Hair follicle cells (HFC) morphology and proliferation potential. HFC were plated in 6-well plates at 10⁴ cells per well and cultured in DMEM with 10% FBS alone (Ctrl) or with 2 ng/ml bFGF. FIG. 1A is a photomicrograph of HFC in the presence or absence of bFGF. FIG. 1B shows cells were cultured in the presence or absence of bFGF and at the indicated times trypsinized, counted and replated at the same density. The cumulative cell number was plotted as mean±SD of three independent experiments (n=3).

FIG. 2A-C: Derivation of SMC from HFC using tissue specific promoters. FIG. 2A shows schematics of lentiviral vectors encoding for ZsGreen under the control of αSMA promoter (P-αSMA) or DsRed under the myosin heavy chain promoter (P-MHC). HFC were transduced with either lentivirus and P-αSMA (ZsGreen+) or P-MHC (DsRed+) cells were sorted by flow cytometry. FIG. 2B shows fluorescence images of P-αSMA (ZsGreen+) or P-MHC (DsRed+) that were cultured in the presence or absence of bFGF (2 ng/ml). Sorted cells were plated in six well plates (10⁴ cells per well) and cultured in the presence or absence of bFGF (2 ng/ml). On day 7, the cells were trypsinized, counted and the cell number was plotted (FIG. 2C) as mean±SD of triplicate samples in a representative experiment (n=3).

FIG. 3A-C: Vascular constructs with P-αSMA cells expressed V-SMC markers. P-αSMA cells were embedded in fibrin hydrogels and cultured around 6-mm mandrels for 2 weeks to form rings. FIG. 3A shows hematoxylin and eosin (H&E) staining showing uniform distribution of P-αSMA cells within fibrin hydrogels (bar=100 μm). Immunostaining for αSMA and calponin (bar=20 μm) is shown in FIGS. 3B and 3C respectively.

FIG. 4: HFC surface marker profile. Flow cytometry with antibodies for mesenchymal stem cell specific markers as indicated. Representative flow cytometry plots from three independent experiments are shown.

FIG. 5A-G: HFC demonstrated adipogenic and chondrogenic differentiation potential. HFC were cultured in adipogenic (A-D) or chondrogenic (E-G) differentiation medium for two weeks. FIG. 5A shows RT-PCR data for adipogenic markers aP2 and PPARγ. Oil Red O staining is shown for HFC that were treated with adipogenic (FIG. 5B) or control (FIG. 6C) medium. FIG. 5D shows a higher magnification of FIG. 5B. FIG. 5E shows RT-PCR data for chondrogenic markers Sox9 and Col II. Type II collagen Immunostaining of HFC pellets is shown for cultured with chondrogenic (FIG. 5F) or control (FIG. 5G) medium. Cells stained with secondary antibody only served as negative control. All samples were counterstained with DAPI to visualize cell nuclei. Representative images are shown from one out of three independent experiments.

FIG. 6A-E: HFC demonstrated osteogenic differentiation potential. HFC were cultured in osteogenic differentiation medium for two weeks. FIG. 6A shows RT-PCR data for osteogenic markers alkaline phosphatase (ALP), Runx2, osteonectin (ON) and osteocalcin (OC). Alkaline phosphates activity is shown for HFC that were treated with osteogenic (FIG. 6B) or control (FIG. 6C) medium. von Kossa staining is shown for HFC that were cultured in osteogenic (FIG. 6D) or control (FIG. 6E) medium for four weeks. Representative images are shown from one out of three independent experiments.

FIG. 7A-D: HFC demonstrated myogenic differentiation potential and force generation ability. HFC were cultured in myogenic differentiation medium for one week. FIG. 7A shows RT-PCR data for αSMA, smoothelin and SM22. FIG. 7B shows immunostaining for αSMA and calponin. Cells stained with secondary antibody only served as negative control. All samples were counterstained with DAPI to visualize the nuclei. Representative images are shown from one out of three independent experiments. HFC were cultured in the presence of bFGF (2 ng/ml) for 5 days when they reached 85-90% confluence. At that time the cells were trypsinized and embedded in fibrin that was allowed to polymerize in 24-well plates to form disks. One hour after polymerization, the gels were detached from the walls and allowed to compact in the presence of FBS alone or supplemented with bFGF (2 ng/ml) or TGF-β1 (2 ng/ml). At the indicated times, gels were photographed and their area was measured using Image J software. FIG. 7C shows the ratio of gel area at the indicated times over the initial area was plotted as % of initial hydrogel area over time. HFC were cultured in DMEM with 10% FBS alone or supplemented with bFGF (2 ng/ml) or TGF-β1 (2 ng/ml) for 5 days. At that time the cells were embedded in fibrin hydrogels that were incubated with medium of the same composition. The % of initial hydrogel area was plotted over time (FIG. 7D). All values represent the mean±SD of triplicate samples in a representative experiment (n=3). Asterisks (*) denote p<0.05 between the TGF-β1 and FBS treated hydrogels at the same time point. bFGF-treated samples were significantly different (p<0.05) than either the TGF-β1 or FBS treated samples at all times.

FIG. 8A-E: HFC-derived P-αSMA cells decrease αSMA expression in response to bFGF. P-αSMA cells were seeded at 10³ cells/cm² and cultured in the presence or absence of bFGF (2 ng/ml). On day 7, the % of ZsGreen+ cells (FIG. 8A) and mean green fluorescence intensity (FIG. 8B) of ZsGreen+ cells were measured by flow cytometry. All values are the mean±SD of triplicate samples in a representative experiment (n=3). P-αSMA cells were fixed, permeabilized and stained mouse anti-αSMA followed by incubation with Alex Fluor® 647-R-phycoerythin goat anti-mouse secondary antibody. The % of cells and the level of αSMA expression were measured by flow cytometry. FIG. 8C shows % of αSMA+ cells and FIG. 8D shows mean green fluorescence intensity of αSMA+ cells. All values are the mean±SD of triplicate samples in a representative experiment (n=3). P-αSMA cells were cultured in the presence of bFGF (2 ng/ml) for 5 days before embedding in fibrin gels that were allowed to compact in DMEM with 10% FBS alone or supplemented with bFGF (2 ng/ml) or TGF-β1 (2 ng/ml). FIG. 8E shows the % of initial hydrogel area plotted over time. All values represent the mean±SD of triplicate samples in a representative experiment (n=3). Symbols (*) or (#) denote significant difference (p<0.05) between bFGF- and FBS- or bFGF- and TGF-β1-treated hydrogels, respectively, at the indicated time point.

FIG. 9: Vascular constructs with P-αSMA cells demonstrated remarkable vasoreactivity. P-αSMA cells were embedded in fibrin hydrogels and cultured around 6-mm mandrels for 2 weeks to form rings. Vascular reactivity was measured using an isolated tissue bath system. FIG. 9A shows representative graphs of isometric contraction over time in response to the indicated agonists. FIG. 9B shows vascular reactivity (N/g dry tissue weight) in response to KCl (118 mM), endothelin (ET)-1 (20 nM) or U46619 (10⁻⁶ M). All values represent the mean±SD of triplicate samples in a representative experiment (n=3).

FIG. 10: Hair follicle smooth muscle progenitor cells (HF-SMPCs) were seeded on to subintestinal submucosa (SIS) and strain was applied to the SIS. FIG. 10 A, B is a representation of scanning electron micrographs at 24 hours post-seeding. FIG. 10 D, E are presentations of EGFP-labeled HF-SMPCs fluorescence images taken real-time. FIGS. 11H and 11H show staining of actin filaments when strain was applied (H), and when no strain applied (G). FIG. 10 C, F, I show actin staining after disruption of the actin filaments with cytochalasin D, an inhibitor of actin polymerization. FIG. 10L shows percent of cells aligned in with our without 10% strain.

FIG. 11 (A-C): Receptor-mediated contraction of vascular constructs in response to both U-46619 and ET1 (FIG. 11A). FIG. 11C shows relaxation response measured using the vasodilators S-Nitroso-N-acetylpenicillamin (SNAP), a nitric oxide donor, and Y27632, a Rho-kinase inhibitor, which were added following vasoconstrictor-induced contraction. FIG. 11 B,C shows constriction and relaxation for vascular construct for which 10% strain was applied, no strain was applied or which was exposed to cytochalasin D.

FIG. 12: shows constriction and reactivity to U-46619 and ET1, SNAP, Y27632 and KCL for vascular construct for which 10% strain was applied, no strain was applied and native artery (n=7).

### DETAILED DESCRIPTION OF THE INVENTION

The patent or application file contains at least one drawing executed in color. Copies of this patent or patent application publication with color drawing(s) will be provided by the Office upon request and payment of the necessary fee.

The present invention is directed to isolating a composition comprising multipotent stem cells from hair follicle. The cells have a mesenchymal phenotype, yet they have the ability to differentiate into smooth muscle cells.

In one embodiment, the present invention provides isolated multipotent cells from adult human hair follicles displaying morphological characteristics of mesenchymal cells and/or displaying markers characteristic of mesenchymal cells and from which smooth muscle cells (SMCs) or progenitors thereof could be isolated. This was unexpected since contractile cells, particularly, smooth muscle cells from adult tissue are difficult to maintain in culture as compared to cells from neonatal tissue. The SMCs obtained in the present invention were highly contractile and vascular constructs prepared from the SMCs exhibited ultimate tensile strength of up to 1.5 megapascals after 2 weeks in culture or up to 100 kilopascals depending upon the scaffold used to seed the cells.

Further, the ability of enriched mesenchymal cell composition from adult human hair follicle to differentiate into various cell types including adipogenic cells, chondrogenic cells and osteogenic cells was also unexpected since we observed that ovine hair follicle cells which could be coaxed into functional contractile smooth muscle cells, failed to differentiate toward osteogenic, adipogenic or chondrogenic cells.

The present method involves harvesting hair follicles. For example, hair follicles can be released from skin (e.g., skin biopsies) with collagenase treatment or can be obtained by plucking; treated with trypsin and EDTA to release single cell suspension or directly cultured in a suitable culture medium for 1-15 days. The cells are allowed to migrate out from each hair follicle. Hair follicle stem cells can be cultured in a medium with or without FBS in the presence of bFGF (0.1-100 ng/ml, TGF-β (0.1-100 ng/ml) or various combinations of both. Hair follicle stem cells can be cryopreserved in liquid nitrogen.

In one embodiment, a population of hair follicle stem cells enriched for mesenchymal cells can be obtained by selecting cells displaying mesenchymal morphology.

Alternatively, the mesenchymal cells can be selected based on specific markers. The selected cells are and then expanded, or pooled and then expanded. Mesenchymal stem cells (MSCs) may be identified and/or characterized by both the presence of markers associated with specific epitopes identified by antibodies and/or the absence of certain markers as identified by the lack of binding of specific antibodies to such markers. MSC may also be identified by the ability of stem cells to give rise to multiple differentiated progeny. The MSC phenotype can be maintained for at least up to 10 passages. Generally, the cells are passaged upon reaching about 70-90% confluence. Passaging is done by routine techniques using mild protease treatment (e.g., trypsin, collagenase etc). These cells have the potential to differentiate into chondrocytes, adipocytes, osteoblasts, smooth muscle cells, endothelial like cells, cardiomyocytes and myocytes. In one embodiment, the mesenchymal cells are positive for one or more of the markers CD44, CD73, CD90 and CD105. In another embodiment, the mesenchymal cells lack the hemeatopoietic markers CD34, CD45 and/or the endothelial marker CD144. In another embodiment, the mesenchymal cells are positive for CD44, CD73, CD90 and CD105 and negative for CD34, CD45 and CD144.

The hair follicles can be obtained from an individual of any species. In one embodiment, the individual is a human. In another embodiment, the human is an adult. In one embodiment, the adult human is over 18 years of age. In another embodiment, the adult human is over 20, 30, or 40 years of age. The source of hair follicles can be skin biopsies or hair follicles can be obtained by plucking hair from an individual's skin.

The methods of the invention provide for substantially enriched populations of MSC. Thus in various embodiments, MSCs may make up at least 50%, 60%, 70%, 80%, 90% or 95% (and all integers between 50 and 95%) of the total number of cells.

To differentiate the cells toward different cells lineages, the cells are exposed to different growth media. For example, for smooth muscle lineage, hair follicle stem cells can be cultured in DMEM or other mammalian cell culture medium with or without serum in the presence of TGFβ, PDGF, bFGF, BMP4 or combinations thereof. For an endothelial like cell lineage, hair follicle (HF) stem cells can be cultured in EGM or other mammalian cell culture medium with or without serum in the presence of TGFβ, PDGF, bFGF, VEGF, EGF, activin or combination of them. For chondrocyte lineage, HF stem cells can be cultured in Dulbecco's Modified Eagles Medium (DMEM) or other mammalian cell culture medium with or without serum in the presence of 6.25 μg/m insulin, TGF-β1,5 ascorbate-2-phosphate or combinations of them. For adipocyte lineage, HF stem cells can be cultured in DMEM or other mammalian cell culture medium with or without serum in the presence of isobutyl-methylxanthine (IBMX), dexamethasone, insulin, indomethacin or combinations of them. For osteoblast lineage, HF stem cells can be cultured in DMEM or other mammalian cell culture medium with or without serum in the presence of dexamethasone, ascorbate-2-phosphate, β-glycerolphosphate or combination of them. For myogenic lineage, HF stem cells can be cultured in DMEM or other mammalian cell culture medium with or without serum in the presence of dexamethasone, TGF-β1, BMP2, BMP4, or a combination of them. For cardiomyocyte lineage, HF stem cells can be cultured in DMEM or other mammalian cell culture medium with or without serum in the presence of 5-azacytidine, TGF-β1, activin, BMP4, or combination of them.

Following differentiation in culture, the culture may comprise at least about 25%, 50%, 75%, 90%, 95% or more of the desired differentiated cells. The cells thus obtained may be used directly, or may be further isolated, e.g. in a negative selection to remove undesired cells or undifferentiated cells. Further enrichment for the desired cell type may be obtained by selection for markers characteristic of the cells, e.g. by flow cytometry, magnetic bead separation, and the like.

In one embodiment, smooth muscle cells or progenitors thereof can be isolated from the population of mesenchymal cells or mixed population of cells. This method involves selecting an enhancer/promoter which functions in the smooth muscle cells or progenitors thereof. A nucleic acid molecule encoding a marker protein under control of the enhancer/promoter is introduced into the mixed population of cells. Such introduction may be transient or a chromosomal integration. The smooth muscle cells or progenitors thereof are allowed to express the marker protein. The smooth muscle cells or progenitors thereof are separated from the mixed population of cells based on expression of the marker protein.

Promoters suitable for carrying out this aspect of the present invention include, without limitation, smooth muscle α-actin promoter, SM22 promoter, caldesmon promoter, myosin heavy chain promoter, calponin promoter and smoothelin promoter. A nucleic acid molecule encoding a marker protein, preferably a green fluorescent protein (“GFP”), under the control of the promoter is introduced into a mixed population of cells or into an enriched population of cells to be sorted. Mutated forms of GFP that emit more strongly than the native protein, as well as forms of GFP amenable to stable translation in higher vertebrates, are now commercially available and can be used for the same purpose. See U.S. Pat. Nos. 5,491,084, and 5,491,084.

Other suitable marker proteins may be derived from neomycin resistance gene (neomycin phosphotransferase), puromycin resistance gene (puromycin N-acetyl transferase), and hygromycin resistance gene (hygromycin phosphotransferase). When included in plasmid DNA, these genes will make the cells resistant to neomycin, puromycin, and hygromycin, respectively. When cells are cultured in antibiotics, only those with the antibiotic resistance marker will survive, and those surviving cells can be recovered.

Various methods are known in the art for introducing nucleic acid molecules into host cells. These include: 1) microinjection, in which DNA is injected directly into the nucleus of cells through fine glass needles; 2) dextran incubation, in which DNA is incubated with an inert carbohydrate polymer (dextran) to which a positively charged chemical group (DEAE, for diethylaminoethyl) has been coupled (the DNA sticks to the DEAE-dextran via its negatively charged phosphate groups, large DNA-containing particles stick in turn to the surfaces of cells (which are thought to take them in by a process known as endocytosis), and some of the DNA evades destruction in the cytoplasm of the cell and escapes to the nucleus, where it can be transcribed into RNA like any other gene in the cell); 3) calcium phosphate coprecipitation, in which cells efficiently take in DNA in the form of a precipitate with calcium phosphate; 4) electroporation, in which cells are placed in a solution containing DNA and subjected to a brief electrical pulse that causes holes to open transiently in their membranes so that DNA enters through the holes directly into the cytoplasm, bypassing the endocytotic vesicles through which they pass in the DEAE-dextran and calcium phosphate procedures (passage through these vesicles may sometimes destroy or damage DNA); 5) liposomal mediated transformation, in which DNA is incorporated into artificial lipid vesicles, liposomes, which fuse with the cell membrane, delivering their contents directly into the cytoplasm; 6) biolistic transformation, in which DNA is absorbed to the surface of gold particles and fired into cells under high pressure using a ballistic device; 7) naked DNA insertion; and 8) viral-mediated transformation, in which nucleic acid molecules are introduced into cells using viral vectors. Since viral growth depends on the ability to get the viral genome into cells, viruses have devised efficient methods for doing so. These viruses include retroviruses, lentivirus, adenovirus, herpesvirus, and adeno-associated virus.

In one embodiment, the present invention provides a preparation of isolated smooth muscle cells or progenitors thereof, where the smooth muscle cells or progenitors thereof constitute at least 80%, 90%, 95, 99 or 100% (and all integers between 80 and 100%) of the preparation and are contractile.

A further aspect of the present invention is directed to a method of producing a tissue-engineered vascular vessel (TEV). This method involves providing a vessel-forming scaffold material onto which or within which smooth muscle cells or progenitors thereof can be seeded. In one embodiment, the scaffold material can be a fibrin mixture containing fibrinogen and thrombin. In another embodiment, the material may be commercially available subintestinal submucosa.

The vessel-forming scaffold is molded into a tubular shape. For example, the fibrin mixture is molded into a fibrin gel having a tubular shape. The fibrin gel having a tubular shape is incubated in a medium suitable for growth of the cells under conditions effective to produce a tissue-engineered vascular vessel. In one embodiment, the materials for making the scaffold (such as fibrinogen, thrombin and the like) are derived from an autologous source (such as the patient's blood).

The strength of the fibrin gel adhesive component may depend on the final concentration of fibrinogen. Higher fibrinogen concentrations can be achieved by increasing the mixing ratio of the typical 1:1 (thrombin:fibrinogen) mixture of the present invention to a 1:5 mixture. The cells in the vessel-forming fibrin mixture are preferably at a concentration within the vessel-forming fibrin mixture of about 1 to 4×10⁶ cells/ml.

The vessel-forming fibrin mixture of the present invention is molded into a fibrin gel having a tubular shape. When cells compact a fibrin gel in the presence of an appropriate mechanical constraint, a circumferential alignment of fibrils and cells results, which resembles that of the vascular media. This alignment characteristic is important in the development of functionality. Mechanical function is dependent on structure, interactions of cells, and extracellular matrix (alignment), as well as composition. Function is also important in the remodeling of the tissue-engineered vasculature vessels. Their structure-function relationship provides a template for the vessel as remodeling occurs.

Molding of the fibrin mixture is preferably carried out in a silastic tube with an inner mandrel. Fibrin gel has the ability to become aligned near a surface as the gel is formed or within the gel as it compacts due to traction exerted by entrapped cells. The use of a central mandrel during gelation increases circumferential alignment of the smooth muscle cells as well as the matrix. The use of a mandrel also provides a large stress on the smooth muscle cells which induces secretion and accumulation of extracellular matrix that enhances the stiffening component of the construct.

During development of the tissue-engineered vasculature of the present invention, it may be desirable to pulse the vessel constructs to modulate growth, development, and structure and/or function of the vessels. When the fibrin vessel constructs are pulsed, there is an inhibition of longitudinal compaction of the construct. In the case of adding a continuous rhythmic pulsation, an increase in cellular alignment perpendicular to the applied force may be achieved. The increased radial alignment created from pulsation may be the limiting factor of the longitudinal compaction.

Pulsing may be achieved by applying force directly to the inner lumen of the tissue-engineered vessel constructs. For example, a roller pump may be used to pass liquid through the inner lumen of the vessels in a pulsating manner. Alternatively, the inner mandrel used in molding the vessel constructs may be connected to a pneumatic pulsation device. In some instances pulsation may have a desirable effect on the structure and/or function of the vessel. In other instances, pulsation may have a detrimental effect on the desired characteristics (structure and/or function) of the vessel.

A suitable medium of the present invention is comprised of M199, 1% penicillin/streptomycin, 2 mM L-glutamine, 0.25% fungizone, and 15 mM HEPES. A growth additive may also be added to the medium suitable for growth. A suitable growth additive is comprised of 50 μg/ml ascorbic acid, 10-20% FBS, 10-20 μg/ml aprotinin or 0.5-2.0 mg/ml EACA, 2 μg/ml insulin, 5 ng/ml TGFβ1, and 0.01 U/ml plasmin. In addition, a growth hormone may be included in the growth additive. Suitable growth hormones include, VEGF, b-FGF, PDGF, and KGF. Preferably, the growth medium is changed every 2-3 days.

Endothelial cells may be seeded to the interior of the tissue-engineered vascular vessel by removing the inner mandrel and seeding the cells to the interior lumen of the vessel. Cells may also be added to the outer surface of the vessels during molding. Suitable cells to be seeded to the outer surface of the vessel include, in a preferred embodiment, fibroblasts. Alternatively, specific organ cells may be seeded to the outer surface of the tissue-engineered vascular vessel of the present invention.

The tissue-engineered vascular vessel of the present invention may also be comprised of a fibrin gel scaffold combined with a porous scaffold to enhance vascular grafting. When the same fibrin gel containing a uniform distribution of cells is used in conjunction with other highly porous scaffold materials, there may be many synergistic benefits of this composite fibrin gel scaffold. There are all the benefits of the fibrin gel plus the addition of early interim strength and early incorporation of other factors that may typically not be produced until later in development (elastin). Thus, the fibrin gel of the present invention can be used with any porous scaffold, such as decellularized elastin or poly lactic-glycolic acid (“PLGA”) to further enhance the benefits and applicability of the fibrin gel vascular grafts. A preferable porous scaffold to be combined with fibrin gel to enhance vascular grafting is decellularized elastin. Another preferable porous scaffold to be combined with fibrin gel to enhance vascular grafting is PLGA.

Smooth muscle cells are known to rapidly degrade fibrin via secretion of proteases. Thus, it is desirable to prevent this degradation during the development of the tissue-engineered vessel of the present invention. Degradation of fibrin in the vessel of the present invention can be controlled through the use of protease inhibitors. A suitable protease inhibitor of the present invention is aprotinin. In one embodiment of the present invention, up to 200 μg/ml of aprotinin is added to the fibrin mixture to modulate fibrin degradation. Preferably, about 20 μg/ml of aprotinin is added to the fibrin mixture to modulate fibrin degradation.

Yet another aspect of the present invention is directed to a tissue-engineered vascular vessel containing a gelled fibrin mixture having fibrinogen, thrombin, and the preparation of isolated smooth muscle cells or progenitors thereof as described above. The gelled fibrin mixture has a tubular shape.

In another embodiment, the smooth muscle cells are seeded on to SIS as follows. A cell suspension of SMCs is added on to SIS and allowed to grow in an appropriate medium. After a day, the cells are contractile. The cells migrate into the SIS. If no strain is applied, a compaction of the SIS is observed indicated by the appearance of folds. However, compaction can be controlled by applying unidirectional strain to SIS, which results in the alignment of the SMCs in the direction of the applied force. The strain can be between 5 and 20% meaning that the SIS is stretched to increase its length by 5 to 20% of the original. In various embodiments, the strain is 5, 6, 7, 8, 9, 10, 11, 12, 13, 14, 15, 16, 17, 18, 19 and 20%. The strain can be applied by applying clamps at the ends or other equivalent means. This results in alignment of the cells in the direction of the applied force. In one embodiment, strain and relaxation can be alternated. To make a tissue engineered vessel (TEV), the SIS comprising aligned SMCs can be joined at the ends (such as by suturing). This can be done while the SIS is wrapped around a mandrel. The mandrel can be removed and then endothelial cells can be seeded on the inside of the hollow structure. Once flow is initiated through the structure, the endothelial cells tend to align along the flow of the fluid.

The tissue-engineered vascular vessel of the present invention is suitable as an in vivo vascular graft. In vivo vascular grafts of the tissue-engineered vascular vessels of the present invention may be made in animals. In a preferred embodiment, the vessel is used as a vein graft in a human being.

A tissue-engineered vascular vessel for a particular patient involves providing a vessel-forming scaffold and the preparation of isolated smooth muscle cells or progenitors described above, at least one of which is autologous to the patient. The vessel-forming scaffold is molded into a tubular shape. The scaffold having a tubular shape is incubated in a medium suitable for growth of the cells under conditions effective to produce a tissue-engineered vascular vessel for a particular patient. The tissue-engineered vascular vessel can then be implanted into the particular patient.

The scaffold material of the tissue engineered vessels can be selected based on the intended use. For example, if the TEV is to be grafted in place of a vein, then a fibrin based scaffold would be appropriate since we observed that such TEVs exhibit ultimate tensile (the force at which the vessel breaks or snaps) strength similar to that exhibited by veins. On the other hand, it the TEV is to be grafted in place of an artery, the scaffold should be such that it exhibits ultimate tensile strength similar to that of arteries. A suitable scaffold for use in arterial grafts is the SIS.

The present invention shows that hair-follicle stem cells can be used for cardiovascular tissue regeneration. Accordingly, HF-SMPCs may be used for engineering vascular grafts, heart valves, or cardiac patches to treat life-threatening cardiovascular disease using autologous cells from a readily accessible source. In addition to SMPC, HF cells may be able to differentiate toward other lineages such as cardiomyocytes or endothelial cells providing cell populations with great potential for the development of cardiovascular therapies. The tissue engineered vesicles using fibrin can be used for venous grafts as these are able to generate constriction similar to that of veins while the tissue engineered vesicles using SIS can be used for arterial grafts as these are able to generate constriction similar to that of arteries.

These aspects of the present invention are further illustrated by the examples below.

### EXAMPLES

The following examples are provided to illustrate embodiments of the present invention, but they are by no means intended to limit its scope.

### Example 1

### Isolation of Multipotent Cells from Human Hair Follicles

This example describes the isolation of multipotent mesenchymal cells from adult human hair follicles.

### Materials and Methods

### Isolation and Cultivation of HFC

Human hair follicle cells (HFC) were isolated as described previously⁹. Briefly, hair-follicle containing full-thickness skin biopsies from the scalp of two donors (41-yr old female and 69-yr old male) were obtained from the Cooperative Human Tissue Network (CHTN, Philadelphia, Pa.). The investigation conforms with the principles outlined in the Declaration of Helsinki.

After extensive washes with PBS (phosphate buffered saline, EMD) containing antibiotic-antimycotic (GIBCO), the tissues were trimmed to remove underlying fat tissue, cut into 2×4 mm pieces and subsequently digested with 0.5% Collagenase Type I (Invitrogen, Carlsbad, Calif.) at 37° C. with occasional agitation. After 4 hr of digestion, single hair follicles were released from the full-thickness skin using fine forceps, filtered through 40 μm cell strainer (BD Biosciences, San Jose, Calif.) and washed extensively with PBS. Then hair follicles were transferred each in a well of a 96-well plate (BD Biosciences) and cultured in 100 μl of DMEM (Gibco, Grand Island, N.Y.) supplemented with 10% FBS (Gibco) to allow for cell migration onto the tissue culture plastic. Cells that originated from the bulge region were visually identified as epidermal keratinocytes, while cells migrating from the dermal sheath or papilla had the morphological appearance of mesenchymal cells. The wells populated with cells originating from the dermal sheath or papilla were selected, pooled and expanded.

### Cloning of Tissue Specific Promoters

Smooth muscle alpha actin promoter (P-αSMA) or myosin heavy chain promoter (P-MHC) was PCR amplified from human genomic DNA using the following primers (P-αSMA: forward primer: ACAACAATCGATAACAGCTGGTCATGGCTGTA (SEQ ID NO.: 1);

reverse primer: TGTTGTACCGGTGCATGAACCCAGCCAAATCC (SEQ ID NO.: 2); and for P-MHC:
forward primer: ACAACAATCGATAGCCTGTGCGGAGCAGCTCA (SEQ ID NO.: 3);
reverse primer: ACAACAGCTAGCGATGGAGAGCTCGGATCTGA (SEQ ID NO.: 4), where the underlined sequences represent the restriction sites ClaI (ATCGAT), AgeI (ACCGGT) or NheI (GCTAGC). The promoter sequences for P-αSMA or P-MHC include 1438 by or 2452 by upstream and 29 by or 53 by downstream of the transcriptional start site (TSS) and were ligated into a promoterless lentiviral vector upstream of ZsGreen (P-αSMA) or DsRed (P-MHC) between ClaI and AgeI for P-αSMA, and ClaI and NheI for P-MHC restriction sites, respectively.

### Flow Cytometry and Immunostaining

HFC were harvested, resuspended in PBS and fixed with 4% paraformaldehyde (RT, 10 min). After three washes in PBS, cells were permeabilized in 0.1% triton-X-100 (RT; 10 min; Fisher Scientific), washed once in PBS and blocked with 2% BSA/PBS (10⁶ cells per 100 μl; RT, 30 min). HFC were then incubated with mouse anti-human alpha-actin antibody (1:100 dilution, RT; 30 min; Serotec) in blocking solution (0.01% triton-X-100 in 1% BSA/PBS). For immunostaining for surface markers the cells were blocked with 1% BSA/PBS (10⁶ cells per 100 μl; RT, 30 min) without fixation or permeabilization. The following antibodies were used CD34, CD44, CD45, CD73, CD90, CD105, CD144, Flk-1 (1:100 dilution in blocking solution; RT; 30 min; BD Biosciences). After three washes in PBS the cells were incubated with Alexa fluor 488-conjugated goat anti-mouse antibody (1:100 dilution; RT; 30 min in dark; Invitrogen) or Alexa Fluor® 647-R-phycoerythrin conjugated goat anti-mouse (1:100 dilution; RT; 30 min; Invitrogen), washed once with PBS and processed for flow cytometry. HFC stained only with secondary antibody served as negative control. Immunostaining and gel compaction assays were performed by methods known in the art.

### Adipogenic and Osteogenic Differentiation of HFC

The differentiation of HFC into adipocytes, chondrocytes and osteoblasts was performed as known in the art (see Zuk et al., Tissue Eng. 2001; 7(2):211-228, the method of differentiation is incorporated herein by reference).

### Contractility and Mechanical Properties of Tissue Equivalents

After two weeks in culture, the length and diameter of cylindrical tissue equivalents were measured using an electronic digital caliper. Then the tissues were released from the mandrel and mounted on two stainless hooks in an isolated tissue bath and incubated in Krebs-Ringer solution. The tissues were continuously bubbled with 94% O₂, 6% CO₂ to obtain a pH of 7.4, a Pco₂ of 38 mmHg, and a PO₂>500 mm Hg at 37° C. Each construct was mounted on stainless steel hooks through the lumen, one was fixed, and the other one was connected to a force transducer. Tissues were equilibrated at a basal tension of 1.0 g and constant length for 30-60 min. After equilibration, potassium chloride (KCl, 118 mM) or the thromboxane A2 mimetic U46619 (10⁻⁶ M) was added to the tissue bath and isometric contraction was recorded using a PowerLab data acquisition unit and analyzed with Chart5 software (ADInstruments, Colorado Springs, Colo.).

For measuring mechanical properties, tissue equivalents were mounted on the force transducer and stretched incrementally until they broke, yielding the ultimate tensile strength (UTS) and break length of each tissue. The initial tissue length corresponds to the length under a passive tension of 1.0 g. Broken constructs were dehydrated with series of ethanol washes, air dried and weighted. UTS was normalized by the dry weight of each construct and expressed in units of Newton per gram of dry tissue weight (N/g dry weight). The elastic modulus was calculated as the slope of the linear part of the length-tension curve.

### Statistical Analysis

Data were expressed as mean±standard deviation and statistical significance (defined as p<0.05) was determined using Student's t-test.

### Results

Isolation of Cells from Individual Hair Follicles

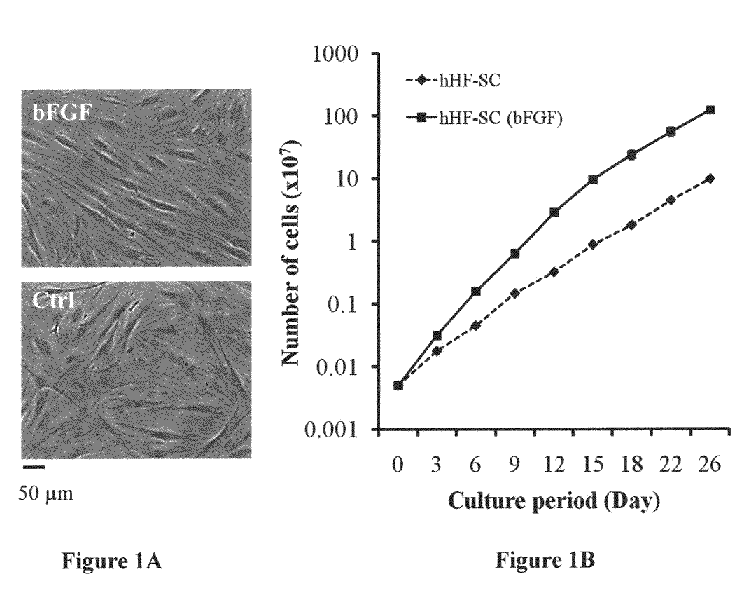

After collagenase digestion, hair follicles were released from the skin, washed extensively and individual follicles were placed each in a well of a 24-well plate to allow for cell migration onto the tissue culture plastic. The migrating cells from individual follicles were either epidermal keratinocytes or mesenchymal cells but some wells contained both cell types. In this study, only mesenchymal cells that were isolated with cloning rings, pooled from several follicles of a 41-yr old female or a 69-yr old male donor were cultured in the presence of absence of bFGF as indicated. As expected from previous studies with mesenchymal stem cells from other sources, in the presence of bFGF (1 ng/ml) hair follicle cells (HFC) appeared smaller, spindle-shaped (FIG. 1A) and proliferated faster, yielding 12-fold increase in cell number after three weeks in culture (FIG. 1B). For this reason HFC that were used in this study were routinely subcultured in bFGF containing medium.

### HFC Displayed the Surface Markers and Multi-Lineage Differentiation Potential of Bone Marrow Mesenchymal Stem Cells

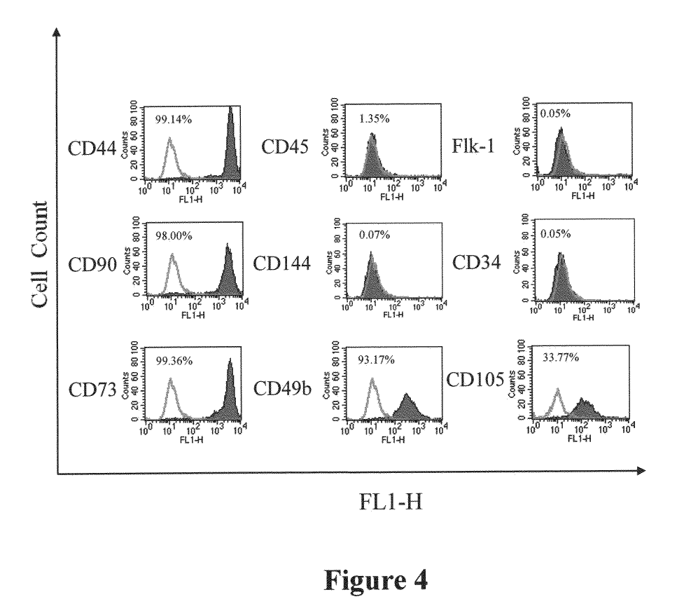

We investigated whether HFC expressed surface markers that characterize bone marrow mesenchymal stem cells. As shown in FIG. 4 HFC displayed nearly the same molecular profile as bone marrow mesenchymal stem cells (BM-MSC). Specifically, HFC expressed high levels of CD44, CD73, CD90 and CD105, but lacked CD34, CD45 or CD144. This result suggested that HFC might display the multi-lineage differentiation potential that characterizes BM-MSC.

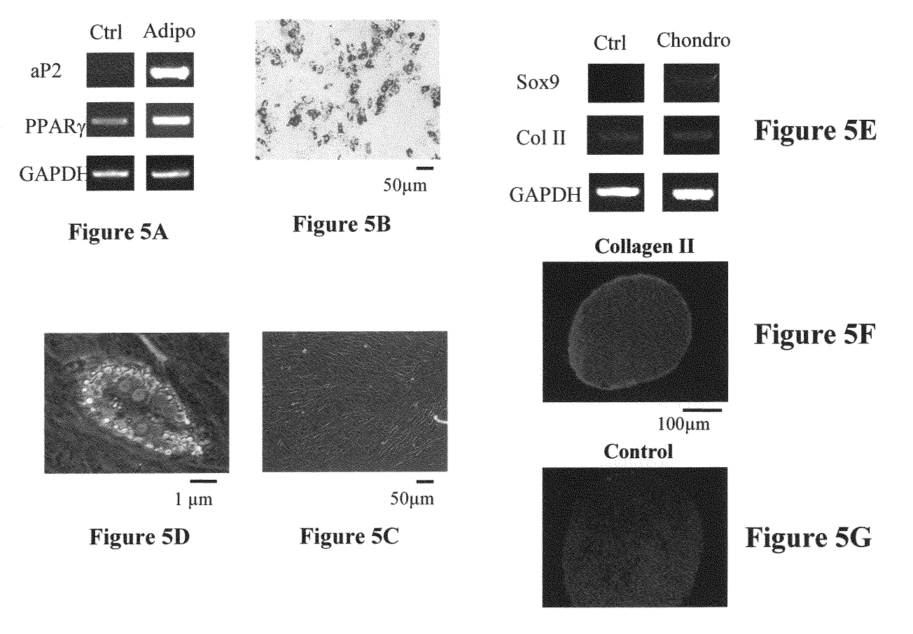

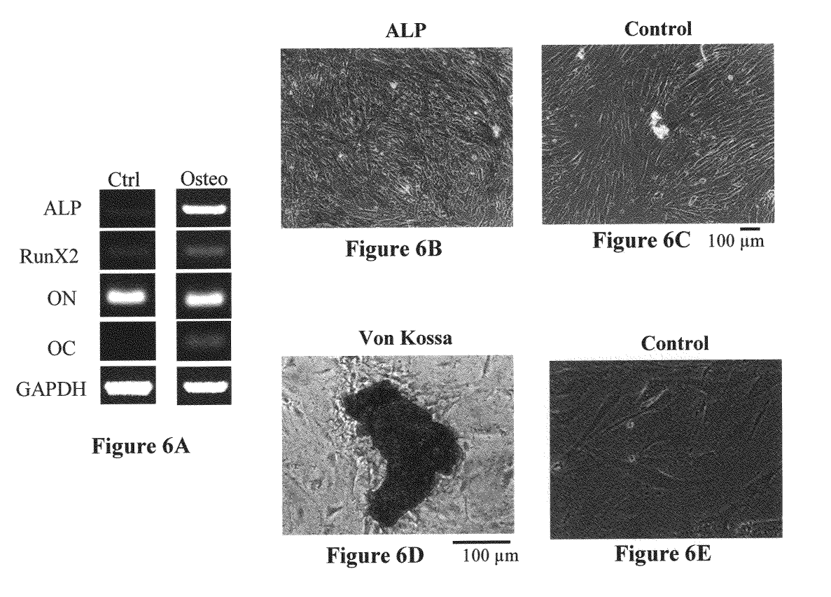

Further, we examined whether HFC could differentiate into fat, bone or cartilage cells under appropriate differentiation conditions. We found that under conditions that promote adipogenesis, HFC expressed adipocyte-specific transcription factors aP2 and PPAR-γ2 (FIG. 5A) and accumulated triglycerides that were visualized by staining with Oil Red O (FIG. 5B-D). Under conditions that are known to promote chondrogenic differentiation (cell pellet cultured in chondrogenic differentiation medium), HFC expressed the genes for collagen II and Sox9 (FIG. 5E) and immunostaining confirmed collagen II expression at the protein level (FIG. 5F, G). In the presence of osteogenic medium, HFC expressed osteogenic markers such as alkaline phosphatase, Runx2, osteonectin (ON) and osteocalcin (OC) (FIG. 6A). Immunostaining demonstrated alkaline phosphatase activity in differentiated (FIG. 6B) but not in control cells (FIG. 6C) and von Kossa staining revealed calcium deposits (FIG. 6D, E). These results suggest that HFC contain multipotent progenitor cells closely resembling BM-MSC in terms of the surface marker profile and multi-lineage differentiation potential.

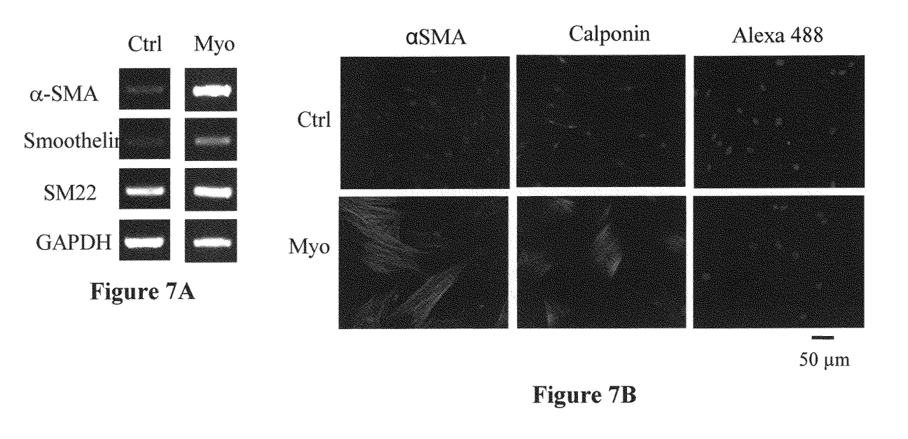

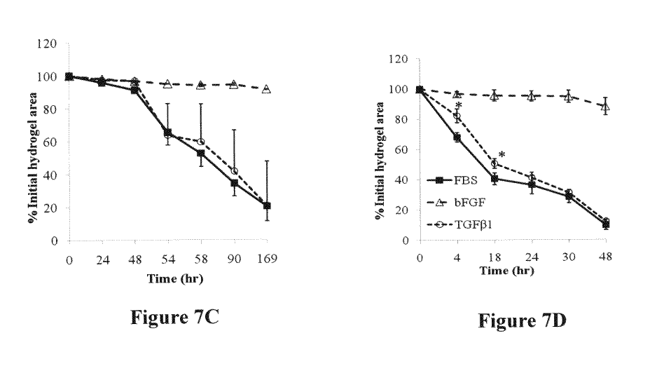

We also examined whether HFC were able to differentiate into smooth muscle cells (SMC). To this end, the cells were cultured in myogenic differentiation medium containing 2 ng/ml of TGF-β1, a cytokine that has been shown to promote differentiation of embryonic stem cells or bone marrow mesenchymal stem cells into mature contractile SMC. Under these conditions, HFC increased expression of SMC markers such as αSMA, smoothelin and SM22 (FIG. 7A). In agreement, immunofluorescence microscopy showed that only in the presence of myogenic medium HFC expressed high amount of αSMA and calponin that were organized in a network of well-defined fibers (FIG. 7B). Note that to enable visualization of control cells the exposure time was much longer (723 ms) compared to cells cultured in myogenic medium (160 ms).

Force Generation by HFC: Effects of bFGF and TGF-β1

An important property of smooth muscle cells is their ability to generate force, which has been previously correlated with expression of αSMA. Since the mRNA and protein levels of αSMA were low in bFGF-containing growth medium and high in TGF-β1 containing differentiation medium, we examined the effect of bFGF or TGF-β1 on the ability of HFC to generate force. To this end, HFC were cultured in the presence of bFGF (2 ng/ml) for 7 days before they were embedded (10⁶ cells/ml) in fibrin hydrogels and overlaid with medium containing 10% FBS alone or supplemented with bFGF (2 ng/ml) or TGF-β1 (2 ng/ml). An hour after gelation the hydrogels were released from the walls and gel compaction was monitored by measuring the area of each gel at the indicated times. As shown in FIG. 7C, following a 48 hr delay, HFC contracted fibrin hydrogels significantly to ˜20% of their initial surface area within ˜7 days (169 hr) when incubated with FBS or TGF-β1. The time to compact to 50% of the original area was approximately 60 hr (t₅₀%=60 hr). Interestingly, treatment with bFGF abolished fibrin contraction, suggesting that bFGF adversely affected the ability of HFC to develop a contractile phenotype.

We also examined whether pre-treatment of HFC with bFGF or TGF-β1 influenced HFC contractility. To this end, HFC were cultured in the presence of bFGF, TGF-β1 or FBS alone for a week and then embedded in fibrin hydrogels and incubated under the same conditions for the indicated times. Interestingly, compaction of fibrin hydrogels prepared with cells that were pre-treated with TGF-β1 occurred much faster (t_{50%} ˜15 hr) and without lag-phase reaching less than 10% of their original area within only 48 hr (FIG. 7D). Interestingly, removal of bFGF during pre-treatment (FBS alone) had similar effect, suggesting that bFGF prevented differentiation of HFC towards a contractile SMC phenotype.

### Isolation of Contractile SMC Using Tissue Specific Promoters

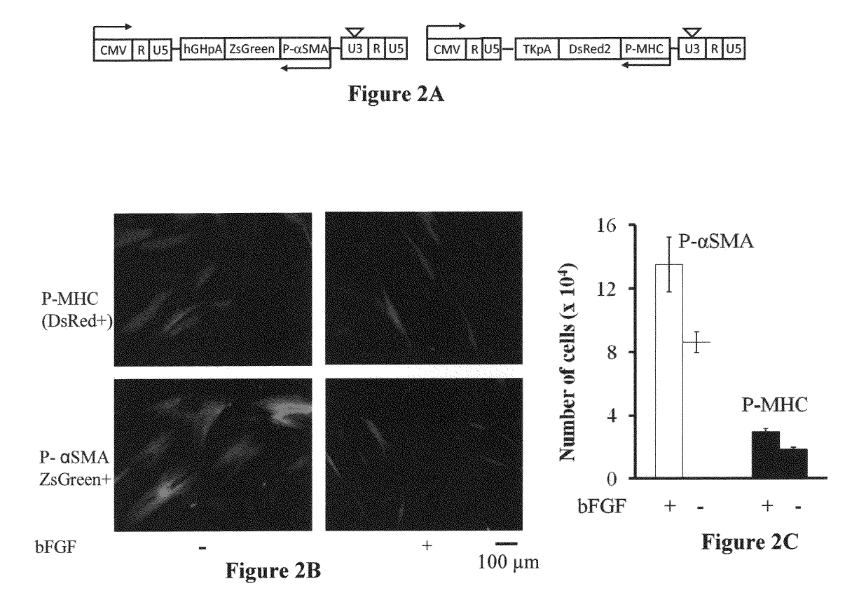

Next we employed smooth muscle specific promoters to identify and purify smooth muscle cells (SMC) from HFC and examined their contractile function. We employed two promoters, namely αSMA and myosin heavy chain (MHC) representing an early and a late marker gene of SMC differentiation. To this end, we generated two lentiviral vectors encoding either for ZsGreen (a bright variant of EGFP) under the αSMA promoter (P-αSMA) or DsRed under the MHC promoter (P-MHC) (FIG. 2A). As bFGF decreased αSMA expression, HFC were cultured in the absence of bFGF for 5-7 days before lentivirus gene transfer and subsequent sorting of green or red cells, respectively.

P-MHC cells (DsRed+, ˜5% of the total population) appeared large and exhibited slow proliferation, consistent with a mature SMC phenotype. On the other hand, P-αSMA cells (ZsGreen+, ˜17% of the total population) were smaller, elongated and proliferated faster, especially in the presence of bFGF (FIG. 2B, C). Due to greater potential for expansion, the rest of the experiments were conducted with P-αSMA cells.

bFGF Decreases αSMA Expression and Contractility of P-αSMA

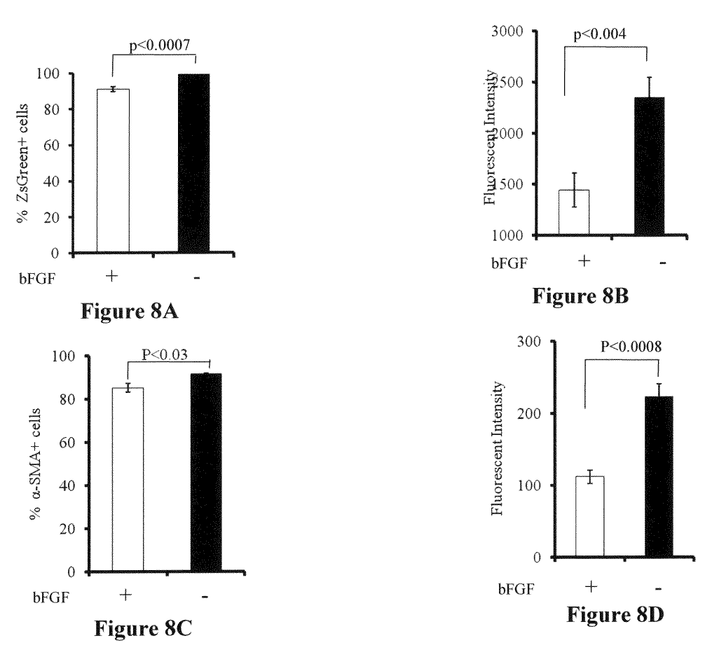

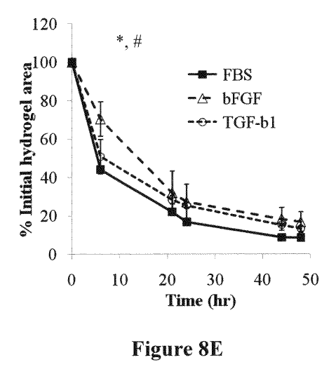

Since bFGF prevented myogenic differentiation of HFC, we examined whether bFGF had an effect on αSMA expression. As shown in FIG. 8A the fraction of HF-SMC carrying active P-αSMA (i.e. ZsGreen+ cells) decreased modestly from ˜99% to ˜91% but the mean fluorescence intensity decreased by ˜40% following bFGF treatment (FIG. 8B). In agreement, flow cytometry using an antibody against αSMA showed that the fraction of αSMA-positive cells decreased somewhat from 92% to 85% (FIG. 8C) and the mean fluorescence intensity decreased by ˜50% (FIG. 8D). In contrast, TGF-β1 did not affect the level of αSMA expression compared to P-αSMA cells cultured in 10% FBS (data not shown). These results suggested that bFGF decreased but did not eliminate αSMA expression in P-αSMA cells.

In agreement with αSMA expression, bFGF delayed but did not abolish fibrin hydrogel compaction. Specifically, within the first 6 hr the hydrogel volume decreased to ˜44% of the original volume for control, ˜50% for TGF-β1-treated and ˜70% for bFGF-treated samples (p<0.05 between bFGF and the other two conditions), respectively (FIG. 8E). On the other hand, the final extent of contraction was not significantly different between control (FBS alone), TGF-β1- or bFGF-treated hydrogels. Interestingly, P-αSMA cells contracted fibrin hydrogels much faster than the original population of HFC (t₅₀%=6 hr for HF-SMC compared to t₅₀%=58 hr for HFC in FBS alone) and even HFC that were pre-treated with TGF-β1 (t_{50%}=18 hr), suggesting that the subpopulation of αSMA+ cells exhibited contractile SMC phenotype.

### P-αSMA Cells Displayed Significant Receptor and Non-Receptor Mediated Vascular Reactivity

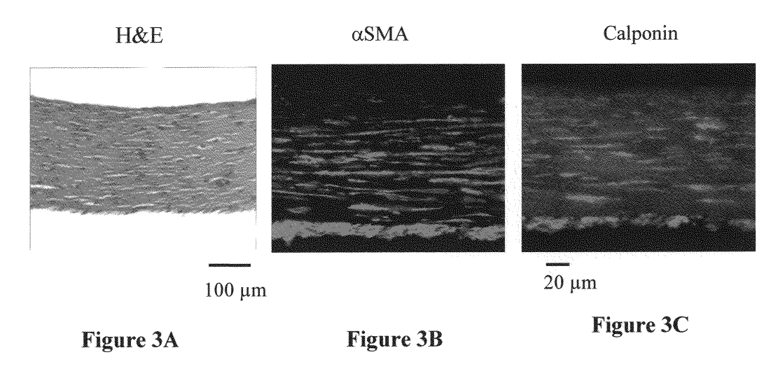

The defining property of mature vascular SMC (V-SMC) is their ability to contract and generate force in response to vasoactive agonists. To examine whether P-αSMA cells exhibited functional properties of mature V-SMC, we measured the isometric tension generated by cylindrical rings of fibrin-based tissue constructs prepared with P-αSMA cells or V-SMC. To this end, cylindrical tissue equivalents were cultured around 6-mm diameter mandrels in the presence of TGF-β1 (2.5 ng/ml) and ascorbic acid (50 μg/ml) for two weeks. At that time, P-αSMA cells or human vascular (V)-SMC tissues compacted significantly reaching wall thickness of 0.22±0.06 mm (n=8) and 0.17±0.04 mm (n=4), respectively. In addition, P-αSMA cells distributed uniformly within the hydrogel (H&E) and stained positive for αSMA and calponin (FIG. 3A-C).

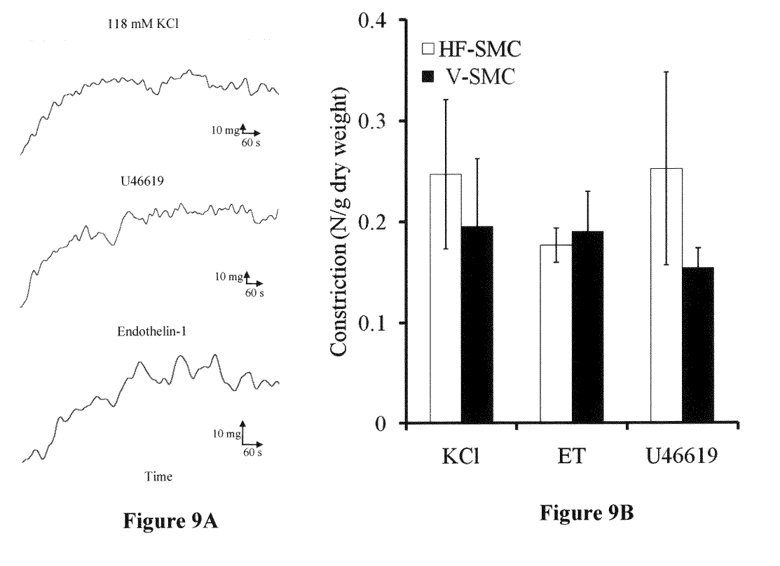

The isometric tension was measured in response to non-receptor and receptor mediated vasoagonists, such KCl (118 mM), endothelin-1 (ET-1, 20 nM) or the thromboxane mimetic U46619 (10⁻⁶M) and representative plots of force vs. time are shown in FIG. 9A. As shown in FIG. 9B vascular constructs prepared with P-αSMA cells (n=4) exhibited similar vascular reactivity to those prepared with V-SMC (n=4), suggesting that P-αSMA cells had developed active pathways of receptor and non-receptor mediated vasoconstriction.

Interestingly, P-αSMA cells based tissue equivalents exhibited significantly better mechanical properties than those prepared with V-SMC as evidenced by the higher ultimate tensile strength (P-αSMA cells: 490.54±41.73 kPa, n=8; V-SMC: 160.20±143.74 kPa, n=4, p<0.03) and Young's modulus (slope of the linear part of the stress-strain curve; P-αSMA cells: 401.77±15.95 kPa, n=8; V-SMC: 157.35±11.43 kPa, n=4, p<0.03). Taken together these results clearly show that P-αSMA cells can generate force when placed in a 3D matrix and contract in the presence of vascular agonists, suggesting that P-αSMA cells display smooth muscle cell phenotype. Therefore, P-αSMA cells were termed hair follicle derived smooth muscle cells (HF-SMC).

### Example 2

### Tissue Engineered Blood Vessels Using SIS as a Scaffold

This example describes tissue engineered blood vessels from smooth muscle cells using SIS as a scaffold material.

### Retroviral Vector Encoding EGFP Under the Control of SMaA Promoter

The rat SMaA promoter was cloned into the promoter-less EGFP reporter vector pEGFP-1 (Clontech, Mountain View, Calif., USA). The SMaA-EGFP sequence from this vector was amplified by high fidelity PCR with forward primer: CCTCTAGACCACGGTCCTTAAGCATGATA (SEQ ID NO.: 5) containing the Xbal site (underlined); and reverse primer: AACTCGAGCCTTACTTGTACAGCTCGTCCATGCCG (SEQ ID NO.: 6) containing the Xhol site (underlined). The PCR reaction was carried out with denaturation for 30 s at 948 C; annealing for 30 s at 94 degrees C., and extension for 90 s at 72 degrees C. The PCR product was subsequently excised with Xbal and Xhol and subcloned into the same sites of the self-inactivating retroviral vector pQCXIX (Clontech), removing the CMV promoter and IRES sequence. The resulting retroviral vector, termed pQ-SMαA-EGFP, encodes EGFP under control of SMαA promoter.

### Retrovirus Production

VSV-G pseudotyped retrovirus was produced by transient transfection of Phoenix-gp packaging cells (kindly provided by Dr. Gary Nolan, Stanford University) with the retroviral vector pQ-SMαA-EGFP and the vesicular stomatitis virus glycoprotein (VSV-G) encoding plasmid. Briefly, Phoenix-gp cells were plated in T-75 tissue culture flask (5_{—}106 per flask) and incubated overnight. The next day, plasmid DNA (10 mg of pQ-SMαA-EGFP and 10 mg of VSV-G) and transfection reagent Fugene 6 (Roche, Indianapolis, Ind., USA) were mixed (1:3, mg:mL) in 800 mL of DMEM without serum or antibiotics. The mixture was incubated at room temperature for 45 min and then added to the culture medium overlaying the packaging cells. The next day, the medium was replaced with fresh culture medium and retrovirus was harvested 24 h later.

Isolation of Smooth Muscle Progenitor Cells from Ovine Hair Follicles

Full-thickness skin was harvested from a newborn lamb under aseptic condition. All procedures and protocols in this study were approved by the Laboratory Animal Care Committee of the State University of New York at Buffalo. The investigation conforms with the Guide for the Care and Use of Laboratory Animals published by the US National Institutes of Health (NIH Publication No. 85-23, revised 1996).

Skin tissue was trimmed to remove underlying fat tissue, cut into 2×4 mm pieces, and subsequently digested with 1 mg/ml of Collagenase Type I (Invitrogen, Carlsbad, Calif., USA) at 378 C with occasional agitation. After 4 h of digestion, single-hair follicles were released form the full-thickness skin, filtered through 40 um cell strainer (BD Biosciences, San Jose, Calif., USA), and washed extensively with PBS. Then, hair follicles were placed each in a well of a 96-well plate (BD Biosciences) and cultured in 100 uL of DMEM (Gibco, Grand Island, N.Y., USA) supplemented with 10% FBS (Gibco) to allow for cell migration onto the tissue culture plastic. Cells that originated from the bulge region were visually identified as epidermal keratinocytes, while cells migrating from the dermal sheath or papilla had the morphological appearance of mesenchymal cells. The wells populated with cells originating from the dermal sheath or papilla were selected, pooled, and expanded. Passage 4 cells were transduced with SMaA-EGFP recombinant retrovirus in the presence of 8 ug/mL polybrene and EGFP+ cells were subsequently sorted using fluorescence-activated cell sorting.

### HF-SMPCs Align in the Direction of Force

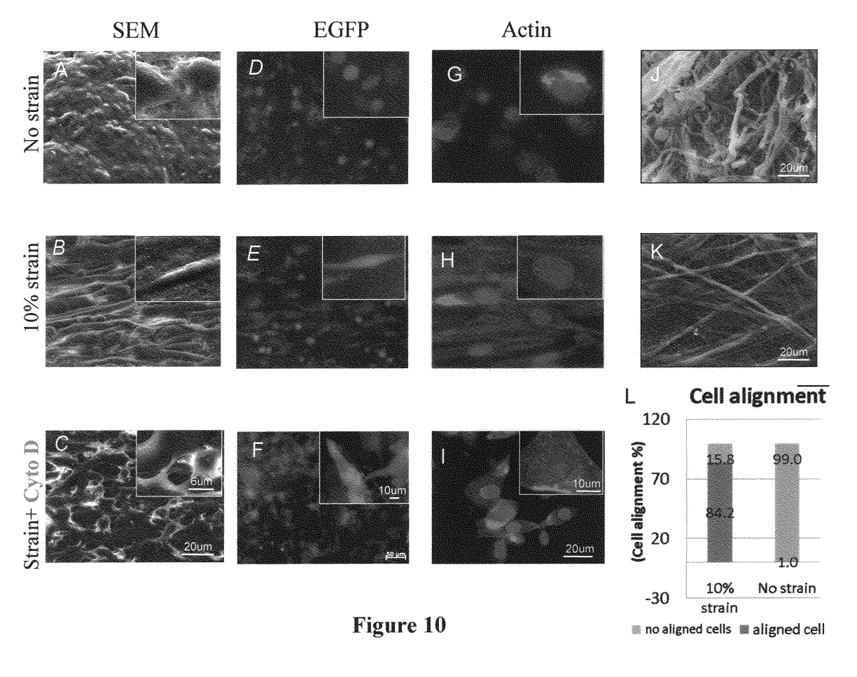

Purified SMCs are seeded on to SIS (Cook Biotech, IN). Upon seeding of HF-SMPCs on SIS, matrix compaction was observed. Cells compacted SIS indicated by folds which extended from the perimeter of the SIS to the center in 24 hrs. Two weeks post-seeding, SIS was highly compacted into a non-uniform mass. To control the compaction, clamps were applied to 2 ends of the SIS sample applying a unidirectional strain. After 2 weeks, a more uniform controlled compaction of the SIS matrix was observed. The force was quantified as a 10% strain, to simulate native arterial physiological levels. Once strain was applied a majority of HF-SMPCs began to elongate and align unidirectionally, in the direction of the applied force, as demonstrated by SEM at 24 hours post-seeding (FIG. 10 A, B). EGFP-labeled HF-SMPCs fluorescence images were taken real-time and could be used to distinguish cells from SIS fibrous structure (FIG. 10 D, E). Additionally, no elongation and alignment of HF-SMPCs was observed immediately after SIS was stretched (data not shown). To understand the mechanism behind the cellular morphological changes in response to force, the actin filaments were stained. The actin filaments were also found to be aligned in the same direction of force (FIG. 10H). Without strain applied, no actin filaments were visualized (FIG. 10 G). The cell alignment and actin filaments were disrupted after cytochalasin D, an inhibitor of actin polymerization, was added (FIG. 10 C, F, I). After strain was applied, SIS fibers were stretched in two major different orientations (FIG. 10 J, K). The directional alignment of SIS fibers was independent of direction of applied force and alignment of cells. 84.2% of the cells were aligned after 10% strain was applied, while almost no cells aligned when strain was not applied (FIG. 10 L).

### HF-SMPC Showed Migration and Extracellular Matrix Molecule Secretion in SIS

After 2 weeks in culture, a majority of HF-SMPCs migrated into SIS. The SIS matrix became more compact and organized. Trichrome collagen staining and Van Hoff elastin staining revealed a significant increase in density of collagen and secretion of elastin by the HF-SMPCs. Notably, collagen I, III and tropoelastin were observed in the high cell-density areas. Moreover, the cells expressed vascular smooth muscle cells specific markers, α-actin, calponin and Myosin heavy chain after the construct was incubated for two weeks.

### HF-SMPC/SIS Vascular Constructs Showed Significant Vascular Reactivity

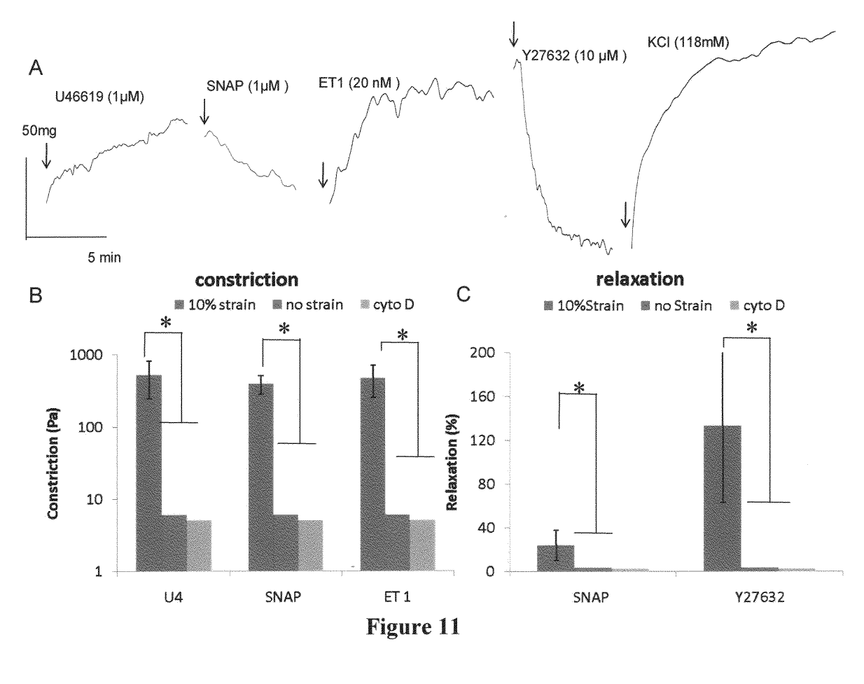

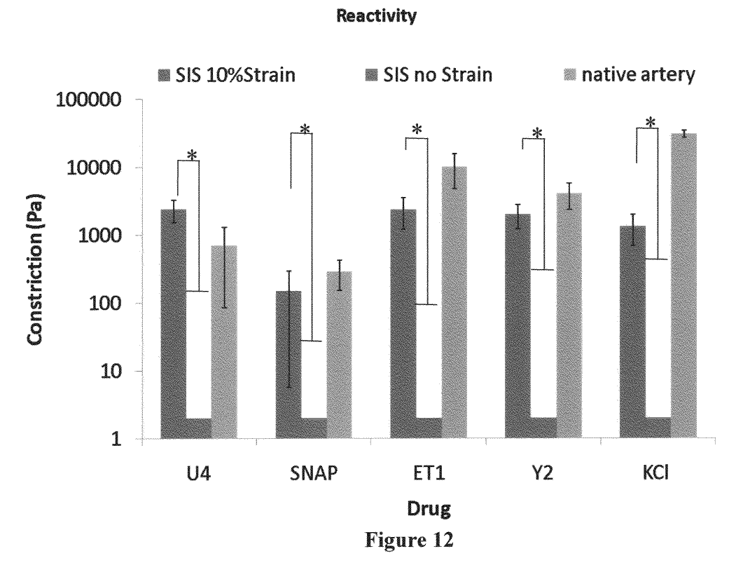

To examine whether HF-SMPCs exhibited functional properties similar to mature SMCs on SIS, we measured the isometric tension generated by vasoactive agents on rectangular stripes of HF-SMPC/SIS vascular constructs as described in Example 1. Receptor-mediated contraction was examined using U-46619, a thromboxane A2 mimetic, (10⁻⁶ M) and Endothelin 1 (ET1) (20 nM). Vascular constructs (n=10) demonstrated significant contraction in response to both U-46619 and ET1 (FIG. 11A). When exposed to potassium chloride (118 mM), a non-receptor-mediated vasoconstrictor, vascular constructs contracted as well. Relaxation response was measured using the vasodilators S-Nitroso-N-acetylpenicillamin (SNAP), a nitric oxide donor, and Y27632, a Rho-kinase inhibitor, which were added following vasoconstrictor-induced contraction. (FIG. 11C) SNAP (10⁻⁶M) relaxed vascular constructs by 25% of the U46619-induced contraction, and Y27632 (10⁻⁵M) generated full relaxation of the Endothelin 1 induced constriction (FIG. 11 B,C). Without strain or with cytochalasin D addition, there were no vascular responses to any of the vasoactive agents. These data indicate that an applied unidirectional force induced cell and actin filament alignment which was essential to vascular reactivity in this construct. The vascular constructs demonstrated significant vascular reactivity as early as 24 hours post-seeding and following 2 weeks in culture. Vascular response increased dramatically over the two weeks (response to U46619 increased from 529.8±286.7 Pa to 2403 Pa; Endothelin 1 from 392.7±223 Pa to 2374 Pa; KCl from 475±249.2 Pa to 1327.4±639.5 Pa; Y27632 from 595.1±537.1 Pa to 2008.7±774.1 Pa, n=6). However, the relaxation to SNAP did not improve significantly (132.5±111.7 Pa to 149.3±143.6 Pa). These vascular constriction and relaxation level are close to ovine native carotid artery (n=7) (FIG. 12). This is the first time that tissue engineered vascular constructs showed vascular contractility to the same order of magnitude as native artery.

Although the invention has been described in detail for the purposes of illustration, it is understood that such detail is solely for that purpose, and variations can be made therein by those skilled in the art without departing from the spirit and scope of the invention which is defined by the following claims.

What is claimed is:

1. A preparation comprising isolated human hair follicle mesenchymal stem cells which have the ability to differentiate into adipocytes, chondrocytes, osteocytes or smooth muscle cells.

2. The preparation of claim 1, wherein the mesenchymal stem cells stain positively for CD44, CD73, CD90 and CD105, but do not stain positively for CD34, CD45 or CD144.

3. The preparation of claim 1, wherein the human hair follicle cells are obtained from adult humans.

4. The preparation of claim 3, wherein the follicle cells are obtained from the dermal sheath or the papilla.

5. A preparation of isolated human hair follicle smooth muscle cells or progenitors thereof, wherein the smooth muscle cells or progenitors thereof comprise at least 90% of said preparation and are contractile.

6. The composition of claim 1, wherein the human hair follicle smooth muscle cells are obtained from an adult human.

7. The preparation according to claim 5, wherein the smooth muscle cells or progenitors thereof comprise at least 95% of said preparation.

8. A method of producing a tissue-engineered vascular vessel comprising:
a) providing a vessel-forming scaffold material selected from the group consisting of i) fibrin mixture comprising fibrinogen and thrombin, and ii) subintestinal submucosa;
b) providing a preparation of isolated smooth muscle cells or progenitors thereof according to claim 5;
c) seeding the isolated smooth muscle cells or progenitors thereof from b) on to the scaffold material from a);
d) shaping the material from c) into a tubular shape; and
e) incubating the tubular shaped material from d) in a medium suitable for growth of the cells under conditions effective to produce a tissue-engineered vascular vessel.

9. The method of claim 8, wherein the step d) further comprises seeding endothelial cells on the inside of the tubular shaped material.

10. The method of claim 8, wherein the scaffold material is subintestinal submucosa and a 5 to 20% strain is applied to the scaffold materials after seeding the isolated smooth muscle cells or progenitors thereof to produce compaction of the scaffold material.

11. The method of claim 11, wherein the strain applied is 10%.

12. The method according to claim 8, wherein the scaffold is fibrin mixture and the tissue engineered vascular vessel has an ultimate tensile strength of up to 0.1 megapascals.

13. The method according to claim 8, wherein the scaffold is subintestinal submucosa and the tissue engineered vascular vessel has an ultimate tensile strength of up to 1.5 megapascal.

14. The method of claim 8, wherein the preparation of smooth muscle cells is from adult human hair follicle.
